(19) United States
(12) Patent Application Publication
(10) Pub. No.: US 2011/0040321 A1
(43) Pub. Date: Feb. 17, 2011

(54) Retrieval Device and Method of Use

(75) Inventor: William A. Cartier, Glenville, NY (US)

Correspondence Address: ANGIODYNAMICS, INC., 14 PLAZA DRIVE, LATHAM, NY, 12110, US

(73) Assignee: ANGIODYNAMICS, INC., Latham, NY (US)

(21) Appl. No.: 12/854,555

(22) Filed: Aug. 11, 2010

Related U.S. Application Data
(60) Provisional application No. 61/232,935, filed on Aug. 11, 2009.

(51) Int. Cl. A61B 17/22

(52) U.S. Cl. 606/200

(57) ABSTRACT

A medical device and method for facilitating safe and effective removal of filters and other related objects from a vascular structure. The device includes a body having a lattice retrieval assembly at a distal end thereof. The lattice retrieval assembly includes an arrangement of intersecting wire members designed to securely engage the hook of a filter or other similar medical device therein. Once the hook of the filter or other similar medical device is captured by the lattice retrieval net, the device is pulled through the sheath thereby causing the retrieval net to collapse upon and completely ensnare the hook of the filter or other medical device to ensure proper removal of the filter from the vascular structure.

This application claims priority to U.S. Provisional Application Ser. No. 61/232,935 filed Aug. 11, 2009.

### FIELD OF THE INVENTION

The present device and method relates to an assembly for retrieving implanted devices. More particularly, the device relates to a retrieval mesh used with implantable devices, such as a filter device, and a method of using the same.

### BACKGROUND OF THE INVENTION

A vast majority of pulmonary emboli originate in the lower portions of the body. For this reason, filters are typically placed in the inferior vena cava to capture potentially fatal blood clots at certain anatomical locations where they may pose less risk of pulmonary emboli for the patient. Conventionally, filters include a hook or retrieval portion; an anchoring portion; and an alignment portion. The anchoring portion is designed to capture and disintegrate blood clots, anchor the filter device, and prevent the filter from migrating downstream; while the alignment portion is designed to provide central alignment of the anchoring portion within the lumen of a vessel and ensures that the device is centered within the vessel. This type of basic filter extends across the vessel and is designed to preferably capture a sufficient percentage of the blood clots, while allowing blood to flow freely through the filter.

A major concern with conventional snare devices is that the loop of the snare device is challenging to manipulate and align properly with the hook of the retrieval portion of filtering devices and causes difficulty with effective removal of the filter. Hence, it is desirable to provide a retrieval tool that is easily positioned about the hook portion of a filter such that the filter is easily captured without the need for additional maneuvering or repositioning of the retrieval tool in order to securely grasp the hook portion of the filter thereby improving removal of the device from the lumen of the vena cava.

### BRIEF SUMMARY OF THE DISCLOSURE

Therefore, a purpose of the device is to provide a retrieval tool that increases the ease and reliability of capturing and retrieving a medical device.

Another purpose is to provide a retrieval tool that has an increased number of contact points which allows for easy removal of a medical device such as a filter or other implantable device.

Still another purpose is to provide a retrieval tool sized for slideable movement with a retrieval sheath such that a retrieval assembly of the retrieval tool is capable of expanding when released from the retrieval sheath and collapsing when housed within the retrieval sheath.

Yet another purpose is to provide a retrieval tool that decreases the level of physician dexterity and amount of skill required for maneuver the device and securely capture the hook or retrieval mechanism of a filter or other implantable device for efficient and proper removal.

In general, the purpose is to provide a device having an improved level of the ability to quickly and proficiently capture and remove retrievable or implanted devices from vessels and other vascular structures.

In one aspect, there is provided a retrieval tool for use with a retrievable or implantable device, such as a blood clot filter. The retrieval tool includes an elongate tubular body housed within an elongate retrieval sheath. The elongate tubular body has a proximal end and a distal end. The distal end of the elongate tubular body includes four arm members each having a first portion welded to the elongate tubular body and a second portion attached to a retrieval assembly. The retrieval assembly securely captures the hook or retrieval mechanism of a filter and aids in safely effectively removing the filter from the vessel or vascular structure.

In another aspect, there is provided a retrieval tool for use with a retrievable or implantable device, such as a blood clot filter that includes a retrieval portion and filter section having a plurality of filter legs, wherein the retrieval tool includes an elongate tubular body housed within an elongate retrieval sheath. The elongate tubular body has a proximal end and a distal end. The distal end of the elongate tubular body includes a retrieval assembly comprising four arm members welded thereto wherein each arm member includes a linear portion and a substantially closed frame portion. The substantially closed frame portion each aid in attaching a retrieval lattice thereto. The retrieval lattice securely captures the hook or retrieval mechanism of a filter and aids in safely and effectively removing the filter from the vessel or vascular structure.

In yet a further aspect, there is provided a method for removing a retrievable or implanted device from a vessel or other vascular structure utilizing the retrieval tool wherein the method includes the steps of at least advancing the retrieval tool to a location of the retrievable or implanted device; pulling back on the retrieval sheath to thereby expose the retrieval assembly; maneuvering the retrieval assembly to securely capture the hook or retrieval mechanism of the retrievable or implanted device within the retrieval lattice; advancing the retrieval sheath over the elongate tubular body such that the retrieval assembly is caused to collapse over the hook or retrieval mechanism of the retrievable or implanted device; and is fully encapsulated within the retrieval sheath; and withdrawing the retrieval tool along with the retrievable or implanted device.

### BRIEF DESCRIPTION OF THE DRAWINGS

FIG. 1 is an isometric view of the retrieval tool.

FIG. 2A is a plan view of the retrieval tool as shown in FIG. 1, positioned within a vessel just prior to capturing a filtering device for removal.

FIG. 2B is an isometric view of the retrieval tool of FIG. 1 in a deployed state with the hook or retrieval mechanism of the filtering device captured by the retrieval assembly.

FIG. 3A is a plan view of the retrieval tool of FIG. 1 partially collapsed and engaging the hook or retrieval mechanism of the filtering device.

FIG. 3B is a plan view of the retrieval tool of FIG. 1 fully collapsed within the retrieval sheath.

FIG. 3C is a plan view of a method detailing the retrieval tool of FIG. 1 and a top portion of the filter enclosed within the retrieval sheath just as the filter begins to collapse and is withdrawn from a vessel within an anatomical structure.

FIG. 4 is an isometric view of another aspect of the retrieval tool.

### DETAILED DESCRIPTION

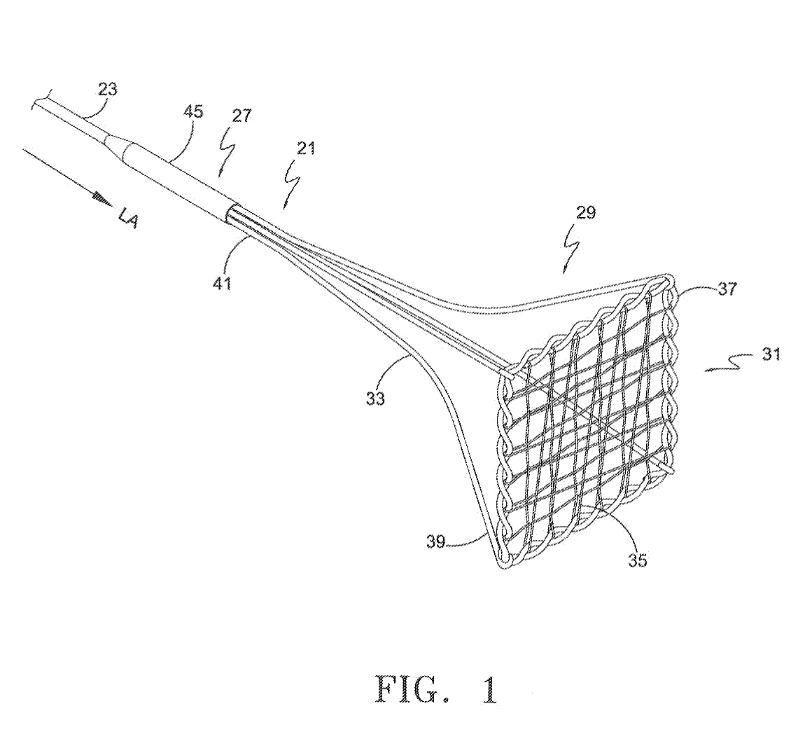

In accordance, FIG. 1 details the retrieval tool (21) without the retrieval sheath. As shown, the retrieval tool (21) comprises an elongate tubular body (23) having a longitudinal axis (LA), a proximal end (not shown), an outer surface (not labeled) and a distal end (27). A retrieval assembly (29) is disposed at the distal end (27) of the elongate tubular body (23). The retrieval assembly (29) includes arm members (33), frame (37) and expandable lattice (31).

The expandable lattice (31) includes an arrangement of intersecting wire members (35) contained within frame (37). Frame (37) is a continuous structure which confines the intersecting wire members (35). The expandable lattice (31) is positioned perpendicular to the longitudinal axis (LA) of the elongate tubular body (23) of the retrieval tool (21). The expandable lattice (31) is attached to the elongate tubular body (23) by arm members (33). Arm members (33) each include a distal portion (39) and a proximal portion (41). Distal portions (39) are attached to frame (37) of the retrieval assembly (29) and the proximal portions (41) are secured to at least a portion of the distal end (27) of the outer surface of the elongate tubular body (23) by welding or any other means known in the art. Shrink tubing (45) may be provided to conceal exposed ends of the welded arm members (33) and to ensure a smooth transition from the welded attachment of the proximal portions (41) of the arm members (33) to the elongate tubular body (23). The shrink tubing (45) allows the retrieval tool (21) to be inserted and withdrawn without causing harm to the anatomical structure from potentially being exposed to and damaged by possible sharp ends of the arm members (33).

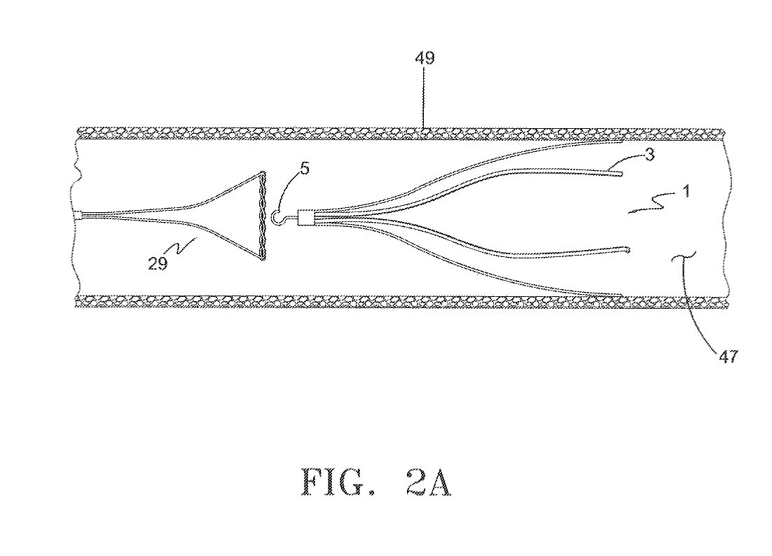

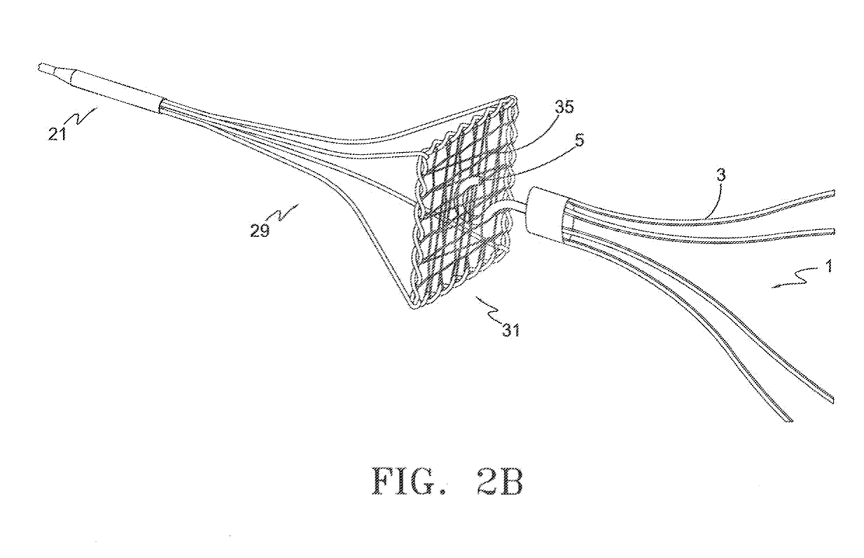

Prior to insertion and retrieval of the filter (1), the retrieval tool (21) is in a first position wherein the retrieval assembly (29) is collapsed and fully enclosed within sheath (not shown). FIG. 2A details the retrieval tool (21) positioned within the lumen (47) of a vascular structure (49) in a second or expanded position just prior to capture of the hook (5) of the filter (1). FIG. 2B shows an isometric view of the hook (5) of the filter (1) fully captured and engaged by the retrieval assembly (29). As shown, the intersecting wire members (35) of the expandable lattice (31) are designed such that the hook (5) of the filter (1) can be very easily captured therein, regardless of the orientation of the hook (5). Over time, endothelial growth and the natural processes of human functions causes the original position of the filter (1) to be altered and become tilted or removed from the centered location within the lumen (47) of the vascular structure (49). The retrieval assembly (29) of the retrieval tool (21) is structured such that the hook (5) of the filter (1) is easily surrounded and restrained by the intersecting wire members (35) of the expandable lattice (31) for secure and effective removal of the filter (1) from the lumen (47) of the vascular structure (49). The large expandable lattice (31) and multiple intersecting wire members (35) of the retrieval assembly (29) provides an increased number of points for making contact with the hook (5) and enables trouble-free retention of the hook (5), even when the filter (1) is oriented at awkward angles or positions within the lumen (47), thereby eliminating the need for withdrawal/reinsertion, additional maneuvering, repositioning or other corrective movement of the retrieval tool (21) prior to withdrawal of the filter (1). As such, the filter (1) can be removed without causing damage to the vessel (49) and without exposing the patient to undue trauma.

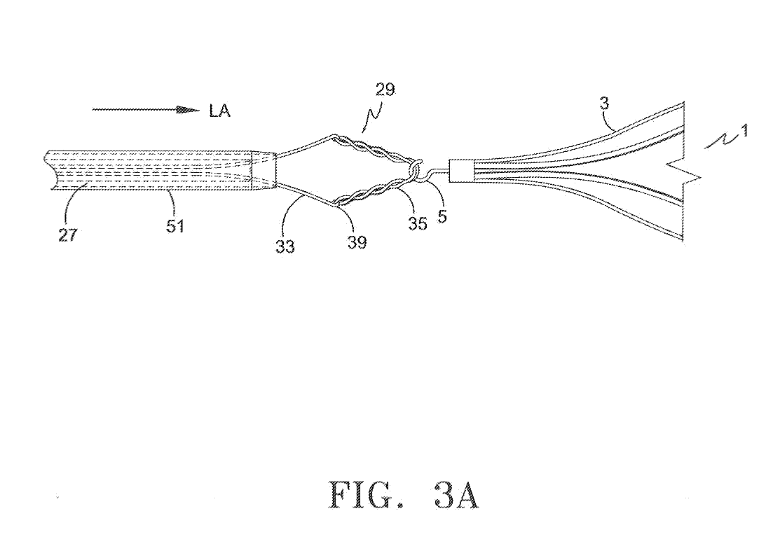

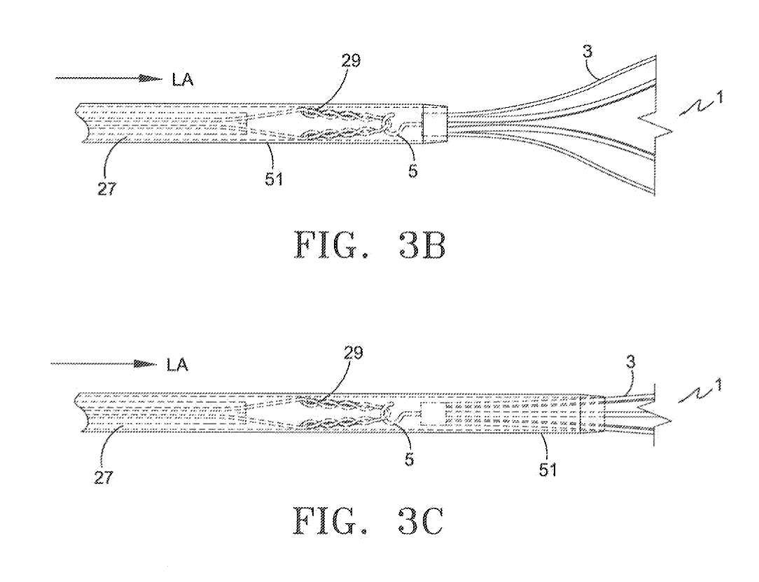

The method of using the retrieval tool (21) to withdraw the filter (1) from the lumen (47) of the vascular structure (49) is detailed in FIGS. 3A-3C. As discussed earlier, after insertion of the retrieval tool (21) into the lumen (47) of the vascular structure (49), the retrieval tool (21) is advanced to the location of the filter (1), the sheath (51) is pulled back to expose the arm members (33) of the retrieval assembly (29) and the expandable lattice (31) is deployed to the second or expanded position (shown in FIG. 2A). The retrieval tool (21) is maneuvered to ensnare the hook (5) of the filter (1) (shown in FIG. 2B) within the intersecting wire members (35) of the retrieval assembly (29). Manual retraction of the retrieval tool (21) while the sheath (51) is held stationary causes the arm members (33) to collapse as they are drawn inwardly toward the longitudinal axis (LA) of the retrieval tool (21), as shown in FIG. 3A. Alternatively, a tugging or pulling force is applied to the proximal end (not shown) of the retrieval tool (21) as the retrieval sheath (51) is simultaneously advanced towards the distal end (27) of the elongate tubular body (23) and over the arm members (33) forcing the arm members (33) to collapse inwardly. Pressure from the arm members (33) passing through the sheath (51) urges the arm members (33) towards one another causing the expandable lattice (31) to collapse. As the arm members (33) begin to move, the expandable lattice (31) of the retrieval assembly (29) starts to fold inwardly towards the distal end (27) of the elongate tubular body (23) of the retrieval tool (21). The expandable lattice (31) becomes elongated and collapses to a cross-sectional area less than an inner diameter of the sheath (51) and securely restrains the hook (5) of the filter (1) within the intersecting wire members (35), see FIG. 3B.

FIG. 3C shows the retrieval tool (21) fully encapsulated by the sheath (51) wherein the arm members (33) are completely collapsed and positioned substantially parallel to the longitudinal axis (LA) of the elongate tubular body (23) of the retrieval tool (21). Once the expandable lattice (31) and hook (5) are entirely received within the retrieval sheath (51), additional tugging or pulling is applied to the proximal end (not shown) of the retrieval tool (21) to collapse the legs (3) of the filter (1) and facilitate withdrawal of the filter (1) from the lumen (47) of the vascular structure (49).

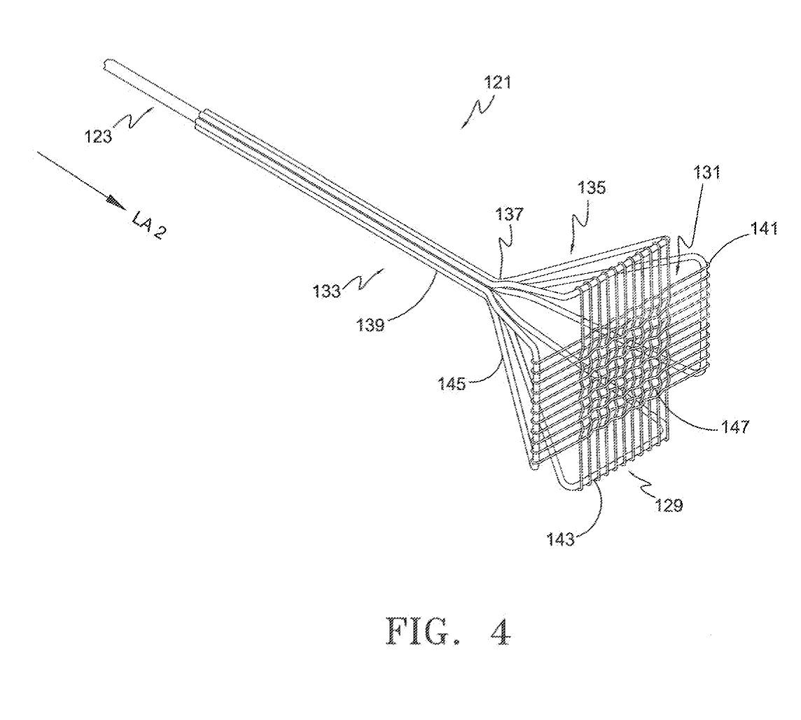

FIG. 4 details an alternate aspect of the retrieval tool (121) depicted without the retrieval sheath. The retrieval tool (121) comprises an elongate tubular body (123) having a longitudinal axis (LA2), and a retrieval assembly (129) disposed distally of the elongate tubular body (123). The retrieval assembly (129) comprises arm members (133) and expandable lattice (131). Arm members (133) having a distal portion that includes frame members (135) positioned distally of the elongate tubular body (123) in opposing directions to one another in a spaced and non-continuous fashion about the expandable lattice (131). Frame members (135) are substantially closed loop frame members.

Arm members (133) further includes a proximal portion comprising linear portions (139) that extend proximally of frame members (135). Linear portions (139) are adjacent to and positioned parallel with the longitudinal axis (LA2) of the retrieval tool (121). Linear portions (139) are further secured to a portion of the outer surface of the distal end of the elongate tubular body (123) of the retrieval tool (121) by welding or any other means known in the art. Shrink tubing (not shown) may also be provided to conceal exposed ends of the welded arm members (133) and to ensure a smooth transition from the welded attachment of the linear portions (139) of the arm members (133) to the elongate body (123). The shrink tubing allows the retrieval tool (121) to be inserted and withdrawn without causing harm to the anatomical structure from potentially being exposed to and damaged by possible sharp ends of the arm members (133).

The expandable lattice (131) is positioned perpendicular to the longitudinal axis (LA2) of the elongate tubular body (123) of the retrieval tool (121) distally of the elongate tubular body (123). The expandable lattice (131) includes an intersecting region (147) having extreme end portions (141) that extend in opposing directions away from the intersecting region (147) towards frame members (135). Extreme end portions (141) are secured also by welding or any other means known in the art to the spaced frame members (135). The intersecting region (147) of the expandable lattice (131) provides an increased number of points for making contact with the hook (5) and enables trouble-free retention of the hook (5), even when the filter (1) is oriented at awkward angles or positions within the lumen (47), thereby eliminating the need for withdrawal/reinsertion, additional maneuvering, repositioning or other corrective movement of the retrieval tool (21) prior to withdrawal of the filter (1). As such, the filter (1) can be removed without causing damage to the vessel (49) and without exposing the patient to undue trauma.

Although the figures detail four arm members (33, 133), the retrieval tool (21, 121) is not limited to four arms and is capable of functioning properly with at least two arm members (33, 133). Arm members (33, 133) and expandable lattice (31, 131) are formed from, but are not limited to, materials such as nitinol, stainless steel, plastic and any other conceivable material that allows the arm members (33, 133) and the expandable lattice (31, 131) to move interchangeably from a collapsed position to an expanded position. The frame members (37, 135) may be triangular, oblong, in the shape of a quadrilateral, circular, oval, tear, diamond, or any other conceivable shape.

Correspondingly, as detailed in FIG. 4, the frame members (135) may comprise a substantially closed triangular loop including a base (143) and two side members (145) that converge at an apex (137). Hence, the linear portions (139) of the arm members (133) extend from the apex (137) proximally therefrom and the extreme end portions (141) extend from one opposed frame member (135) to the other opposed frame member (135) and are welded, respectively, to the base (143) of the opposing frame members (135).

An unlimited number of variations and configurations for the retrieval tool (121) could be realized. The foregoing discussion describes merely exemplary illustrations of the principles and scope of which is recited in the following claims. Those skilled in the art will readily recognize from the description, the claims, and drawings that numerous changes and modifications can be made without departing from the spirit and scope of the retrieval tool (121). Accordingly, the scope of the retrieval tool (121) is not limited to the foregoing specification.

What is claimed is:

1. A medical device for retrieving implanted devices from a vascular structure comprising:
an elongate tubular body having a longitudinal axis, an outer surface, a proximal end and a distal end and a retrieval assembly attached to the distal end of the elongate tubular body and positioned perpendicularly to the longitudinal axis.

2. The medical device of claim 1, wherein the retrieval assembly comprises an expandable member and at least two arm members each having a proximal portion attached to the distal end of the elongate tubular body and a distal portion attached to the expandable member.

3. The medical device of claim 2, wherein the device comprises four arm members.

4. The medical device of claim 2, wherein the retrieval assembly further comprises at least one frame member positioned about and connected to the expandable member.

5. The medical device of claim 4, wherein the expandable member comprises an expandable lattice having an arrangement of interconnecting wire members confined within the at least one frame member.

6. The medical device of claim 2, wherein at least a portion of the arm members are concealed within a shrink tubing.

7. The medical device of claim 2, wherein the expandable member and the at least two arm members are formed from a material selected from the group consisting of nitinol, stainless steel, and plastic.

8. The medical device of claim 4, wherein the retrieval assembly is configured to move interchangeably from a collapsed position to an expanded position.

9. The medical device of claim 5 wherein the at least one frame is selected from the group consisting of a quadrilateral, circular, oval, tear, diamond, oblong and triangle.

10. The medical device of claim 4, wherein each of the at least two arm members comprises the at least one frame member and a linear portion positioned proximally of the at least one frame member.

11. The medical device of claim 2, wherein the retrieval assembly further comprises four frame members each spaced about and positioned opposite another frame member about the expandable member.

12. The medical device of claim 11, wherein the frame member comprises a substantially closed loop.

13. The medical device of claim 12, wherein the substantially closed loop comprises a base and two side members positioned on opposing sides of the base such that the two side members converge at an apex proximally of the base.

14. The medical device of claim 13, wherein the linear portion extends from the apex of the substantially closed loop along at least a portion of the longitudinal axis of the elongate tubular body and is attached to at least a portion of the outer surface of the elongate tubular body at the distal end thereof.

15. The medical device of claim 13, wherein the expandable member further comprises an expandable region having a plurality of extreme end portions extending outwardly therefrom in opposing directions such that each extreme end portion is attached, respectively, to the opposing base member of the substantially closed loop frame member.

16. The medical device of claim 4, wherein the at least one frame member is a continuous frame.

17. A method of retrieving an implanted device from a vascular structure comprising the steps of:
inserting a retrieval device comprising an elongate tubular body having a longitudinal axis, an outer surface, a proximal end and a distal end, having at least two arm members attached to at least a portion of the outer surface of the distal end of the elongate body; and an expandable member connected to a distal portion of the at least two arm members;
advancing the retrieval device to a location of the implanted device;
expanding the at least two arm members and the expandable member such that the expandable member is disposed perpendicularly to the longitudinal axis of the elongate tubular body;
maneuvering the expandable member to ensnare a retrieval mechanism of the implanted device; and
retrieving the implanted device from the vascular structure.
